(19) United States
(12) Patent Application Publication
(10) Pub. No.: US 2017/0202586 A1
(43) Pub. Date: Jul. 20, 2017

(54) COMPOSITE IMPLANT TRIAL

(71) Applicant: DEPUY SYNTHES PRODUCTS, INC., Raynham, MA (US)

(72) Inventors: Christopher CAMPBELL, West Chester, PA (US); Erasmo LOPEZ, Wyndmoor, PA (US); Daniel KWAK, West Chester, PA (US)

(21) Appl. No.: 15/372,534

(22) Filed: Dec. 8, 2016

Related U.S. Application Data
(60) Provisional application No. 62/266,050, filed on Dec. 11, 2015.

(51) Int. Cl. A61B 17/80

(52) U.S. Cl. CPC A61B 17/8052; A61B 17/8033

(57) ABSTRACT

Disclosed is a novel system for trialing implants used in orthopedic surgery. In one embodiment, the invention is directed to a composite trial implant that employs a wireframe with holes for guide wires (K-wires) and fixation screws. The novel trial implant allows a surgeon to determine the correct size, hole configuration, and cross-sectional profile with a simulated device, that permits more rapid and accurate selection of a permanent implant.

### CROSS REFERENCE TO RELATED APPLICATIONS

This application relates to U.S. patent application Ser. No. 62/266,050, filed Dec. 11, 2015; all applications are herein incorporated by reference in their entireties.

### FIELD OF THE INVENTION

In a preferred embodiment, the present invention generally relates to systems and methods for novel composite implant trials used in orthopedic surgery.

### BACKGROUND

Various implants, plates, and screws are commonly used to repair fractures and deformities of bones, during orthopedic surgical procedures. It is common in surgical orthopedic procedures for surgeons to select plate sizes using multiple implants available in the re-sterilized sets to determine the correct length of the needed plate or implant, and in some cases width or depth. These implants are placed and removed onto surgical site, e.g. site of the bone fracture or deformity. The present method suffers drawbacks, however, since re-sterilized implant sets may not include the needed plate or implant or such plate or implant in the correct size or configuration of screw-holes, guide holes (i.e., k-wire holes), etc. In addition, when plates are provided to the surgeon in sterile packaging, the surgeon must determine the correct size of implant prior to opening the sterile packaging. The use of trials (i.e., mock-ups of implants used during the surgical procedure to determine sizing, configuration, etc.), sizing guides, and/or templates is typically used to assist in the implant selection process.

Present, commercially available trials do not adequately meet the clinical needs or economic concerns. Anatomical metal trials are heavy, can be costly to manufacture and do not adequately allow for visualization of screw locations using imaging technology. Anatomical plastic trials, while lighter in weight and more cost efficient, are often considered less durable and do not adequately permit the visualization of screw locations using imaging technology. Further, two-dimensional profile metal or plastic trials are simpler to manufacture and are cost efficient but do not provide anatomical information that surgeons require, and do not adequately permit the visualization of screw locations using imaging equipment.

There is a need for trials/sizing guides for orthopedic surgical procedures that are able to be used to determine anatomical fit information, as well as length and screw locations, while being cost efficient, allow for radiographic visualization and minimize disruption to surgical procedure.

### BRIEF SUMMARY OF THE INVENTION

The invention can be summarized as a composite trial implant composed of a wireframe embedded within a polymer matrix. The wireframe has a spine with one or more insert holes for k-wires or screw holes for fixation or locking screws. The screw holes may be configured to illustrate the screw trajectory that would result from insertion of a screw through the holes. The screw holes may be conical to permit the use of presently commercialized screws that can be inserted at variable angles and/or include locking means.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention can be embodied in different forms and thus should not be construed as being limited to the embodiments set forth herein.

FIG. 1A shows an illustrative embodiment of a wireframe according to the present invention, in cross-section.

FIG. 1B shows an illustrative embodiment of a wireframe according to the present invention, in top plan view.

FIG. 2A shows an illustrative embodiment of wireframe according to the present invention having a circumferential ring, in cross-section.

FIG. 2B shows an illustrative embodiment of wireframe according to the present invention having a circumferential ring, in top plan view.

FIG. 3A shows an illustrative embodiment of a composite implant according to the present invention, in cross-sectional view.

FIG. 3B shows another embodiment of a composite trial implant according to the present invention having a drug-coating layer, in cross-section.

FIG. 4A shows an illustrative embodiment of composite trial implant according to the present invention, in cross-section.

FIG. 4B shows another illustrative embodiment of composite trial implant according to the present invention, in cross-section.

FIG. 5A shows an illustrative embodiment of a trial implant having a non-linear cross-sectional profile.

FIG. 5B shows another illustrative embodiment of a trial implant having a non-linear cross-sectional profile.

FIG. 5C shows an enlarged, cross-sectional view of a guide wire insertion hole, according to an embodiment of the present invention.

FIG. 5D shows an enlarged, cross-sectional view of a screw insertion hole, according to an embodiment of the present invention.

FIG. 6A shows an illustrative guide-wire insertion hole, according to an embodiment of the present invention, having a plurality of flow holes, in perspective view.

FIG. 6B shows an illustrative screw hole according to an embodiment of the present invention, having a plurality of flow holes, in cross-sectional view.

FIG. 7A shows another illustrative screw hole according to an embodiment of the present invention, having a plurality of flow holes, in cross-sectional view, with a screw partially inserted therethrough.

FIG. 7B shows the illustrative screw hole according to the embodiment of FIG. 7A, with the screw fully inserted therein.

FIG. 8 illustrates implant trials in top plan view.

FIG. 9 illustrates various composite trial implants in top plan view and cross-sectional view.

FIG. 10A illustrates an embodiment of an implant trial according to the present invention having a plurality of guide holes and screw holes.

FIG. 10B illustrates the trial implant of FIG. 10A in cross-sectional view.

FIG. 10C illustrates an embodiment of a composite trial implant according to the present invention.

FIG. 10D illustrates the composite trial implant of FIG. 10C in cross-sectional view.

FIG. 10E illustrates an embodiment of an implant trial according to the present invention having a plurality of guide holes and screw holes, and includes indicia providing information useful during a surgical procedure, in top plan view.

FIG. 11A illustrates another composite trial implant according to an embodiment of the present invention, in top plan view.

FIG. 11B illustrates a trial implant according to another embodiment of the invention having indicia, in top plan view.

FIG. 11C illustrates a trial implant according to another embodiment of the invention, in top plan view.

### DETAILED DESCRIPTION OF THE INVENTION

The present subject matter will now be described more fully hereinafter with reference to the accompanying figures and examples, in which representative embodiments are shown. The present subject matter can, however, be embodied in different forms and should not be construed as limited to the embodiments or examples set forth herein.

A novel composite trial composed of at least both a metal wireframe and polymer substrate is disclosed. It has been found that the instantly described composite trial implant (also referred to herein simply as a “trial”) provides improved performance trials used during orthopedic trauma surgical procedures. Trials according to the present invention are better able to communicate anatomical fit information, provide length and screw locations, are more cost efficient than using actual, sterilized implants, permit improved radiographic visualization, and greater reliability during surgical procedures.

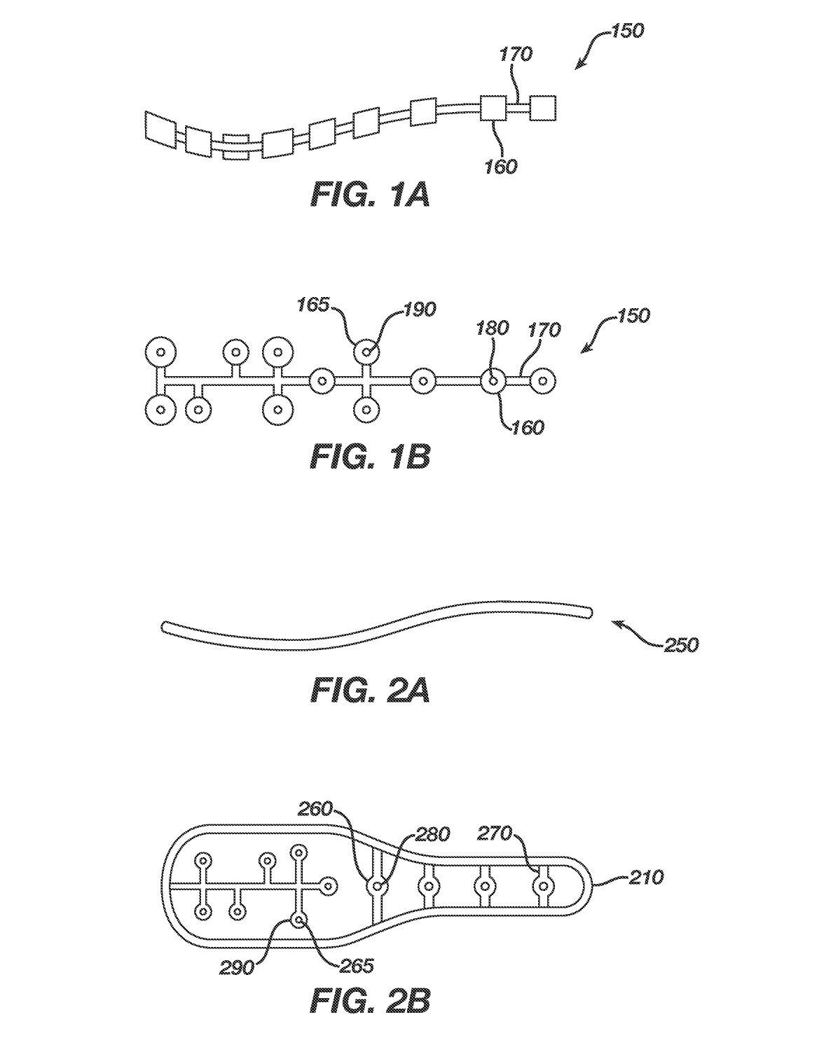

In one illustrative embodiment, the invention includes at least wireframe. As shown in FIG. 1A, the wireframe 150 may be comprised of a plurality of spine sections 170 and holes 160 that can be used as drill guides, for guide wires (e.g. K-wires, or Kirschner wires), or for fixation screws that secure the implant to bone. As show in in FIG. 1B, the wireframe 150 contains screw guides 165 that are cannulated with holes 190 for receiving fixation screws. The wireframe 150 also includes guide wire guides 160 and guide wire holes 180. Exemplary trial implants may include one or both when providing a guide for the implantation of a bone plate.

A wireframe 250 need not be flat, as it may be used to approximate the topology of the bone onto which a bone plate may ultimately be secured, as illustrated in FIG. 2A. As shown in FIG. 2B, the wireframe 250 may include an external wireframe element, such as a circumferential ring 210, and includes one or more spine elements 270, guide wire guides 260, and guide wire holes 280 in the guide wire guides 260. Additional spine elements may support the placement of screw hole guides 290, each having a screw hole 265 therein.

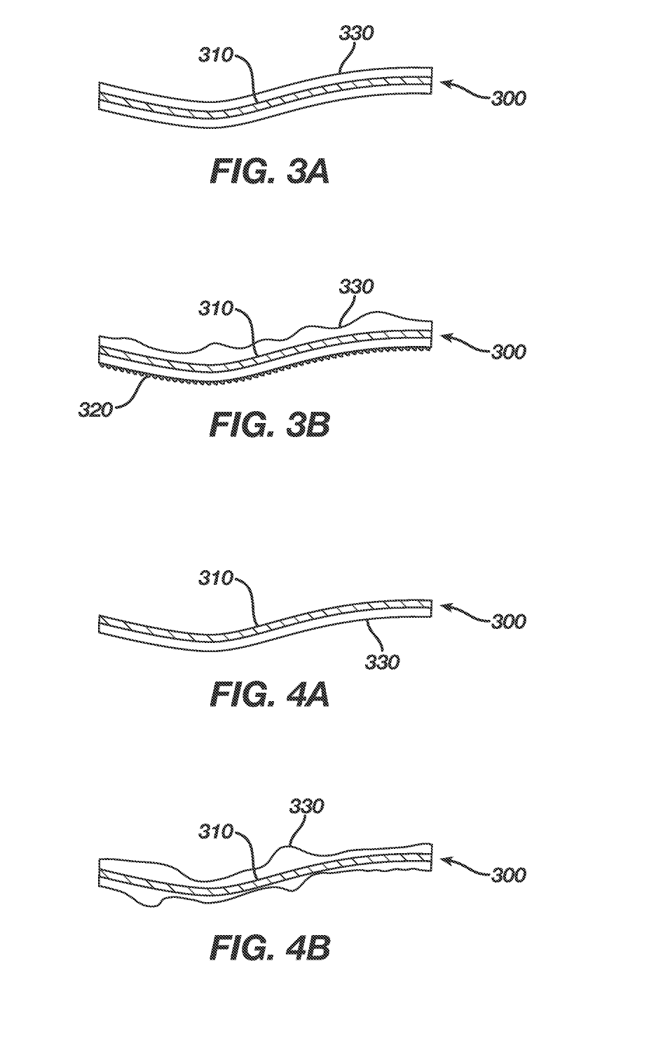

Each composite trial implant will include a wireframe, typically made of metal or other rigid (although preferably flexible) material encapsulated within or partially encapsulated within a solid polymer matrix. As illustrated in FIG. 3A, the wireframe 310 may be embedded within a polymer matrix. Not shown in FIG. 3A is view of the polymer matrix showing that the polymer matrix has a size and shape generally similar to an implant or bone plate that will eventually be secured to the bone. FIG. 3B shows a composite trial implant 300 that has an irregularly shaped top surface 330. The wireframe 310 is fully embedded within the polymer matrix and a pharmaceutically active coating 320 is disposed on a bottom surface of the composite trial implant 300. In some embodiments, the wireframe 310 is radio-opaque to allow the position of the wireframe to be seen in fluoroscopy or x-rays. This feature permits the surgeon to accurately determine the location of the composite trial implant 300 relative to the bone. This may permit the surgeon to insert guide wires using the composite trial implant and then replace the composite trial implant with a permanent bone plate (or other implant) which is then secured to the bone to provide stability to the fractured bone fragments.

With the external wireframe, the outer profile of the implant can be communicated along with screw locations. If the wireframe is made of radio-opaque materials, the outer profile of the wireframe and screw locations can be displayed on an x-ray with the bone. Further, wireframe has the ability to allow for screw trajectories to also be communicated through cannulated holes. The cannulated holes could as be used for a drill guide. With the objective of minimizing surgical disruption, if the holes are cannulated for drilling the surgeon would be able to trial and drill at the same time. The manufacturing method for the wireframes would ideally be 3D printing with additional finishing steps for surface finishing or cannulated holes with more traditional manufacturing methods. This would significantly reduce the manufacturing costs of the wireframes.

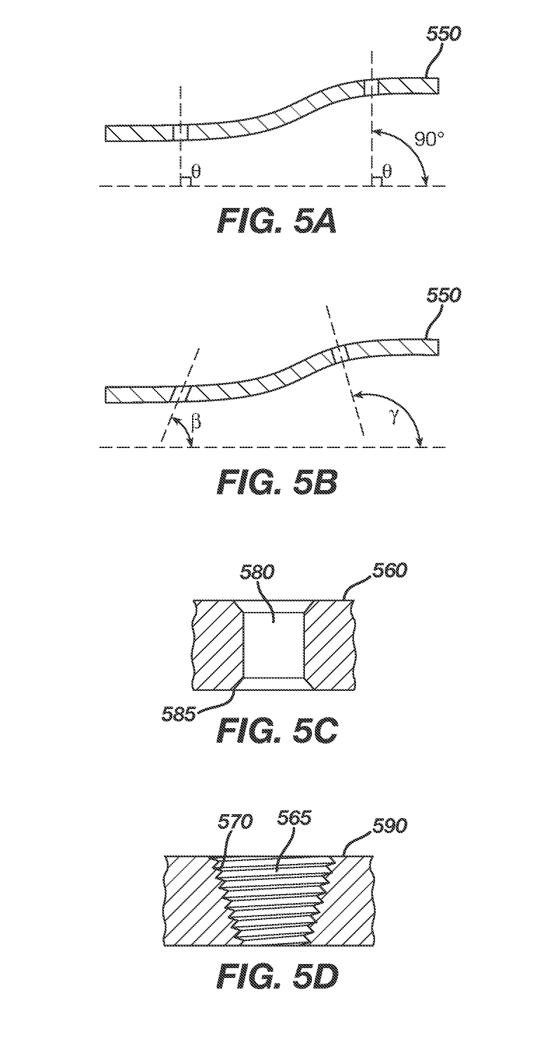

As shown in FIG. 5A, the composite trial implant 550 may have a non-linear cross-sectional profile. Screw holes may be provided that provide a trajectory θ for screws inserted therethrough. In FIG. 5B, screw holes that are not perpendicular to the plane of the composite trial implant may be more or less than 90 degrees, such as angles β and γ. Thus, the present invention permits a more accurate device for surgeons to determine screw trajectories, and even pre-drill holes in the bone, prior to placement of the permanent implant.

The wireframes can be embedded into a polymer matrix/substrate in multiple ways as shown in FIGS. 3A, 3B, 4A, and 4B. Such embodiments may include a single layer where the polymer substrate covers the wireframe only on one side. Embodiments may also include a double layer in which the polymer covers the wireframe top and bottom. Embodiments may also include a variable composite layer which allows for various surface variations to either aid in trial placement or to match the implant anatomical shape. These surface variations can be very expensive in traditionally machined trials but is cost-efficient in an injection molded, 3D printer, or other manufactured method for the polymer matrix. The surface prep composite layer (FIG. 4B), allows for various surface coatings (e.g. antibiotics or pharmaceutical drug) to be applied to polymer matrix; surface coatings are easier on a carbon based polymer chains. This is particularly applicable if the idea of composite structure is extended to implants and coatings can be applied with timed dispersion mechanisms. The polymer matrix can be implemented through various manufacturing methods but ideally suited to injection molding or 3D printing; joining of the wireframe can occur through insert/over molding during injection molding or assembly with or without a bonding agent after the wireframe and polymer matrix have been manufactured separately.

FIGS. 5A and 5B illustrates various methods of hole creation at various angles within the composite trial depending on intended use. Cannulated hole used for K-wire insertion is one method of hole creation in the wireframe. A threaded hole can be created through direct machining in the wireframe or using an Insert that snaps or assemblies into the wireframe. This would allow for the inserts to be produced on a large scale and reduce cost of manufacturing.

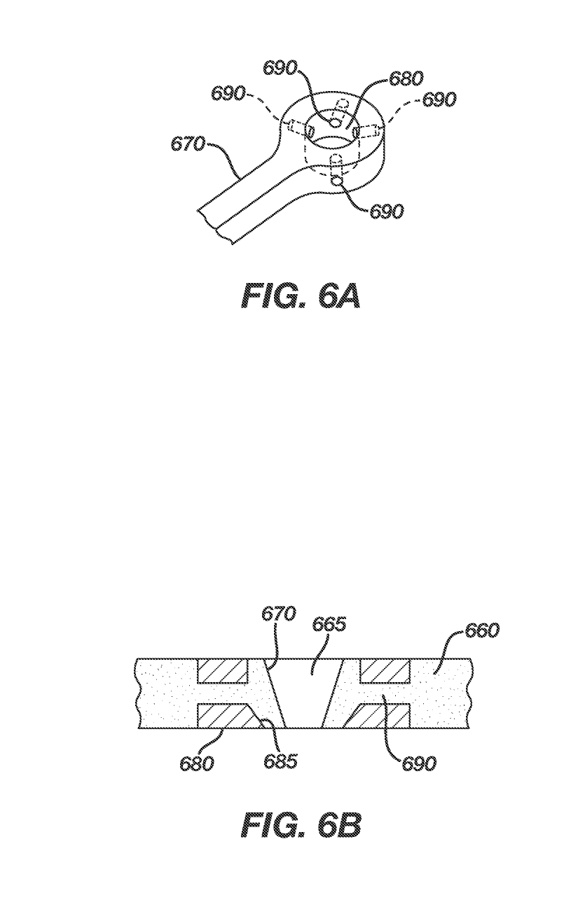

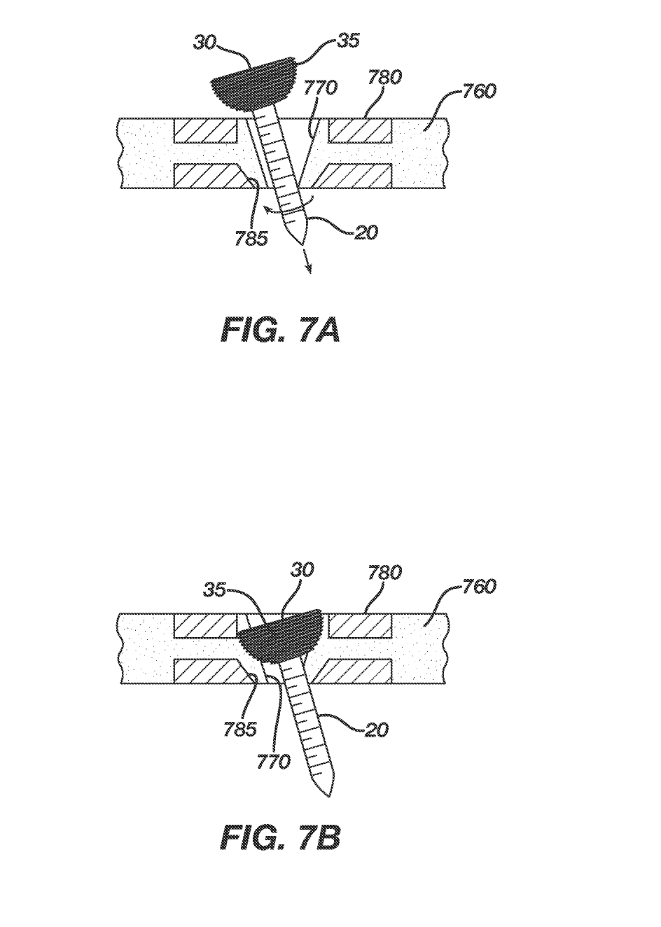

Various methods of snap features are illustrated. As shown in FIGS. 6A and 6B, polymer composite holes 690 can also be created with injection molding of the polymer matrix 660. There would be flow holes 690 in the wireframe that would allow for the polymer to flow into the cannulation 680 and form a straight or tapered hole 670. A screw with a threaded head could be inserted through this polymer hole and the threads on the head of the screw form threads into the polymer. This allows for natural locking due to the thread forming mechanism into the polymer. In addition the wire frame can provide various support functions of the thread including increasing strength and endurance while also providing a stop 685 against screw pull through. This concept of polymer composite hole can be extended into an implant, the polymer matrix would need to be biocompatible (e.g. PEEK) and wireframe would need to be implant grade material. FIGS. 7A and 7B illustrate a screw having a threaded head that can be inserted through the cannulated hole with varying trajectories, permitting the surgeon to create a screw trajectory that best captures bone fragments or provides the greatest stability to the bone being fixed with a plate or implant.

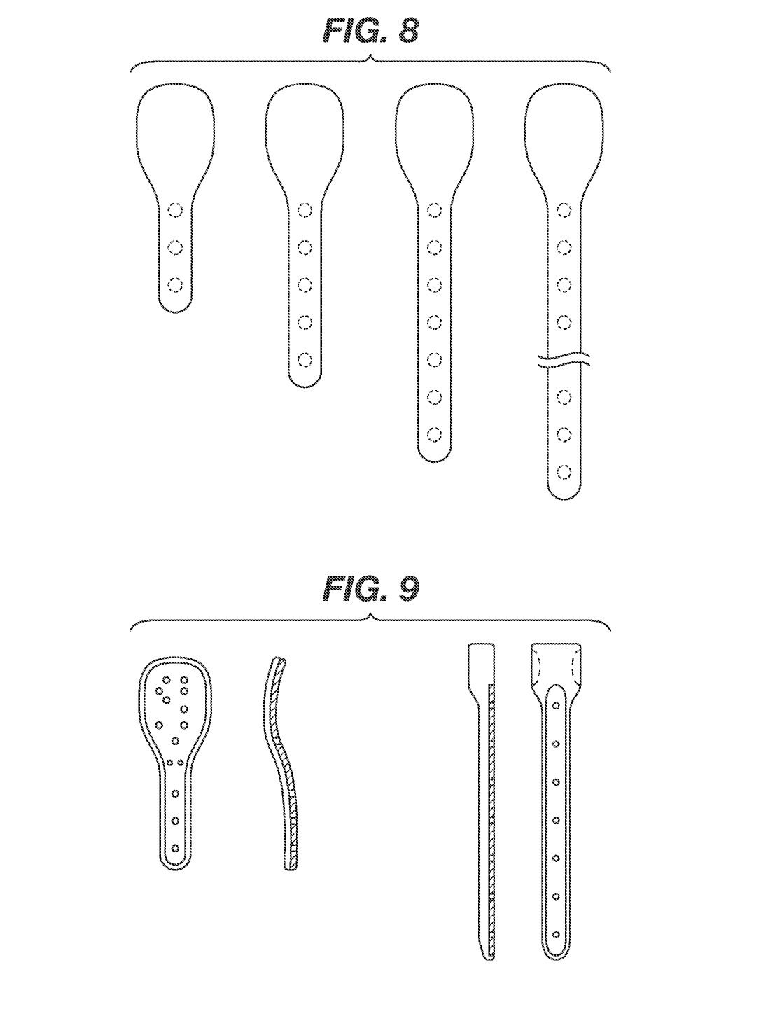

Implants in most systems, especially trauma systems, contain implants that share common features with a few points of variations; primarily the variation is length (shaft holes) but also includes head width, hook depth and extension features. As shown in FIG. 8, the concept of the composite trial supports the trialing of an implant system with a limited number of composite trials. The example shown is an implant family that varies in length from 3 hole to “n” hole. The composite trial allows for connector features to be designed into the polymer matrix so that you would only need two trials to support the entire “n” hole family of implants; there are multiple variations of this concept that could reduce the required composite trials to more or less than 2 trials. The concept is that the composite trial “articular head” would allow for the anatomical fit within the anatomical region while the “length connector” allows for shaft length measurement. This connector concept is shown additionally in FIG. 9.

Most orthopedic/trauma implants have K-wire holes that allow for preliminary fixation during the procedure. As shown in FIG. 9, K-wire holes also may be placed in the “articular head” of the composite trial to allow for “pinning” or securing the location of the desired implant once the trial has been placed at the desired position. Once the K-wires have been inserted, the trial can be removed and the actual implant selected can be placed into the approximate location using the K-wires. This would significantly reduce the surgical implant of trialing during a Trauma procedure. It also could allow for more efficient surgical procedure and reduce the impact of using actual implant to trial during the surgical procedure.

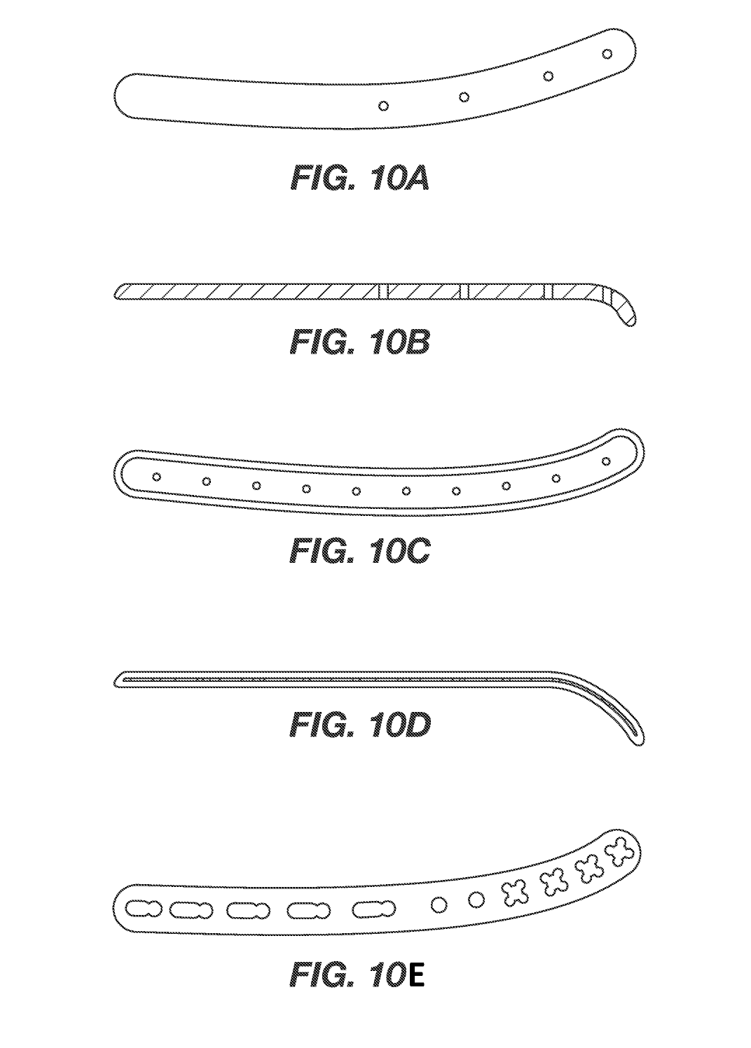

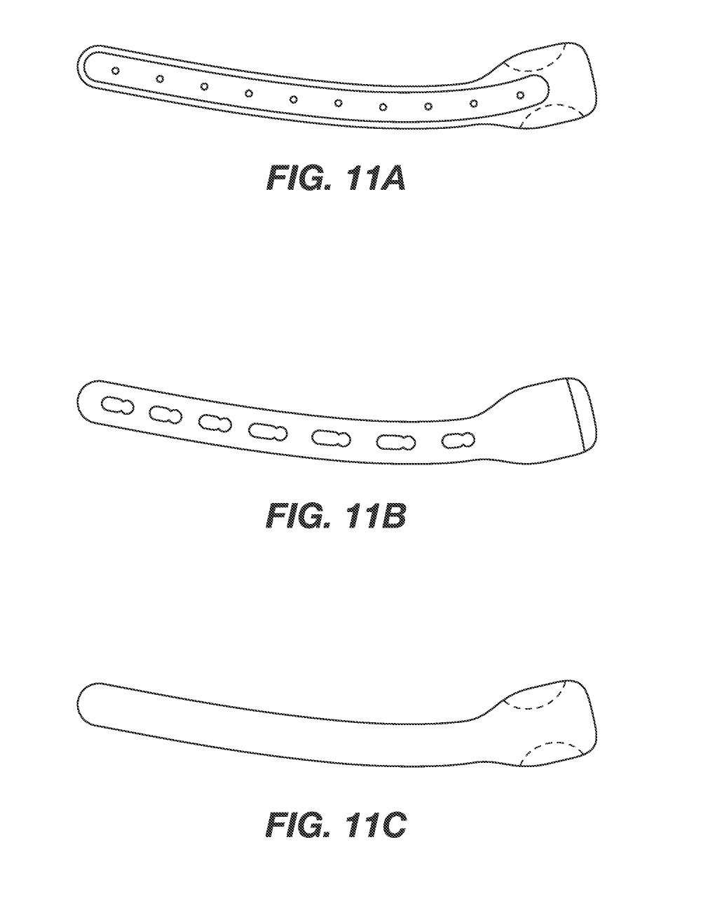

FIGS. 10A-10E illustrate various embodiments for composite trial implants.

Together the composite trial meets the need for trials/sizing guides in orthopedic trauma procedures that are able to communicate anatomical fit information as well as length and screw locations while being cost efficient, allow for radiographic visualization and minimize disruption to surgical procedure.

In addition, as mentioned in the above disclosure, the idea of the composite trial can be extended into a composite implant comprised of a biocompatible and implant grade polymer matrix and wireframe. This could reduce the cost of manufacturing implants while maintaining the clinical strength requirements by optimizing the design characteristic of the composite implant. In addition various surface coatings could more easily be considered given the carbon chain of the polymer; this could allow for various combination drugs between pharmaceutical or biologics depending on the material characteristics and selection.

It should be understood that various changes, substitutions, and alterations can be made herein without departing from the spirit and scope of the invention as defined by the appended claims. It should also be apparent that individual elements identified herein as belonging to a particular embodiment may be included in other embodiments of the invention. Moreover, the scope of the present application is not intended to be limited to the particular embodiments of the process, machine, manufacture, and composition of matter, means, methods and steps described in the specification. As one of ordinary skill in the art will readily appreciate from the disclosure herein, processes, machines, manufacture, composition of matter, means, methods, or steps, presently existing or later to be developed that perform substantially the same function or achieve substantially the same result as the corresponding embodiments described herein may be utilized according to the present invention. Furthermore, all publications, patent applications, patents, and other references mentioned herein are explicitly incorporated by reference in their entirety.

1. A composite trial implant, comprising:
a polymer matrix;
a wireframe at least partially embedded within the polymer matrix;
the wireframe including at least one hole configured to receive a guide wire or a screw.

2. The composite trial implant of claim 1 comprising wherein the hole is a screw hole configured to review a screw in a variable trajectory.

3. The composite trial implant of claim 1 wherein the wireframe comprises a spine having a plurality of holes configured to receive guide wires.

4. The composite trial implant of claim 3 wherein the wireframe comprises a plurality of screw holes depending therefrom.

5. The composite trial implant of claim 4 wherein the screw holes are configured to receive a screw in a variable trajectory.

6. The composite trial implant of claim 1 wherein the polymer matrix comprises PEEK.

7. The composite trial implant of claim 1 having a non-linear cross-sectional profile.

8. The composite trial implant of claim 1 having indicia thereon.

9. The composite trial implant of claim 1 wherein the wireframe is radio-opaque.

10. The composite trial implant of claim of claim 1 comprising a pharmaceutically active coating on at least one side of the polymer matrix.
